(19) United States
(12) Patent Application Publication
(10) Pub. No.: US 2024/0315977 A1
(43) Pub. Date: Sep. 26, 2024

(54) SOFTGEL CAPSULE

(71) Applicant: R.P. Scherer Technologies, LLC, Carson City, NV (US)

(72) Inventors: Karunakar Sukuru, St. Petersburg, FL (US); Qi Fang, St. Petersburg, FL (US); Haitao Li, St. Petersburg, FL (US); Aris Gennadios, St. Petersburg, FL (US)

(21) Appl. No.: 18/575,474

(22) PCT Filed: Jul. 6, 2022
(86) PCT No.: PCT/US2022/036190

§ 371 Date: Dec. 29, 2023

Related U.S. Application Data
(60) Provisional application No. 63/218,579, filed on Jul. 6, 2021.

(51) Int. Cl. A61K 9/48; A61K 47/36; A61L 2/232; A61L 101/34

(52) U.S. Cl. CPC A61K 9/4825; A61K 9/4833; A61K 47/36; A61L 2/232; A61L 2101/34

(57) ABSTRACT

A softgel capsule is provided which can be used with a fill composition including a high alcohol content. The softgel capsule includes a shell composition including a film forming polymer and a plasticizer. The fill composition includes at least about 20 wt. % alcohol. The softgel capsule has a weight loss change of less than about 10% after 30 days, 90 days, 6 months, 9 months or 12 months storage at ambient conditions.

### CROSS REFERENCE TO RELATED APPLICATION

The present application claims priority to U.S. Provisional Application No. 63/218,579 filed on Jul. 6, 2021, the entire content of which is incorporated by reference in its entirety.

### TECHNICAL FIELD OF THE INVENTION

The present invention relates to a softgel capsule for encapsulating a fill composition comprising alcohol. More specifically, the present invention relates to a softgel capsule for encapsulating a fill composition comprising, e.g., at least about 20 wt. % alcohol.

### BACKGROUND OF THE INVENTION

Alcohol is a common ingredient found in various pharmaceutical products and personal care products, such as sanitizers. In pharmaceutical products, ethyl alcohol is commonly used for solubilizing active pharmaceutical ingredients and to enhance the bioavailability of pharmaceutical products. In personal care products, alcohol is commonly used as a sanitizing agent in hand sanitizer gels.

There are a large number of dosage forms in which pharmaceutical products and personal care products are prepared. Soft gelatin capsules (referred to as liquid gels or softgels) are a unique drug delivery system that can provide distinct advantages over traditional dosage forms such as tablets, hard gelatin capsules and liquids. A softgel is a hermetically sealed, one-piece capsule typically with a liquid or semisolid fill. The softgel typically includes two major components, the shell composition and the fill composition. An exemplary shell composition may comprise a film forming polymer such as gelatin. The fill compositions can include a wide variety of vehicles and can be, for example, a solution or a suspension.

Often, a softgel capsule utilizes an alcohol to solubilize a pharmaceutical product. Having alcohol in the fill composition of a softgel has been found to be challenging in preparing a stable product that is suitable for commercialization. Recently, there is a high demand for single use alcohol based hand sanitizers because of the ongoing COVID-19 Pandemic. The current single use dose hand sanitizer is packaged using plastics or plastic/aluminum laminate film pouches to prevent alcohol loss. However, these packaging materials are of environmental concern because they are not biodegradable. There continues to be a need in the art for a softgel suitable for alcohol containing fill compositions and environmentally friendly and acceptable packaging for alcohol based products.

### OBJECTS AND SUMMARY OF THE INVENTION

It is an object of certain embodiments of the invention to provide a softgel capsule comprising a fill composition comprising an alcohol.

It is an object of certain embodiments of the invention to provide a softgel capsule comprising a fill composition comprising an active agent solubilized in an alcohol.

It is an object of certain embodiments of the invention to provide a softgel capsule comprising a fill composition comprising a sanitizing agent (e.g., a hand sanitizer).

It is an object of certain embodiments of the invention to provide a method of preparing a softgel capsule as disclosed herein.

It is an object of certain embodiments of the invention to provide a method of treating a disease or condition with a softgel capsule as disclosed herein.

It is an object of certain embodiments of the invention to provide a method of sanitizing a surface (e.g. a body surface) with a fill composition of a softgel capsule disclosed herein.

One or more of the above objects of the invention and others may be met by certain embodiments of the invention.

In certain embodiments, the invention is directed to a softgel capsule comprising a shell comprising a film forming polymer and a plasticizer, and a fill composition comprising at least about 20 wt. % alcohol.

In certain embodiments, the invention is directed to a softgel capsule comprising a shell comprising a film forming polymer and a plasticizer, and a fill composition comprising at least about 20 wt. % alcohol wherein after 30 days, 90 days, 6 months, 9 months or 12 months storage at ambient conditions, the softgel capsule has a weight loss change of less than about 10%.

In certain embodiments, the invention is directed to a method of treating a disease or condition comprising administering a softgel comprising an active agent as disclosed herein to a subject or patient in need thereof.

In certain embodiments, the invention is directed to a method of preparing a softgel as disclosed herein comprising encapsulating a fill composition comprising at least about 20 wt. % alcohol in a shell comprising a film forming polymer and a plasticizer.

In certain embodiments, the invention is directed to a method of sanitizing a surface comprising dispensing an alcohol composition from a softgel disclosed herein and applying the composition to the surface.

### BRIEF DESCRIPTION OF THE DRAWINGS

The present invention is illustrated by way of example, and not by way of limitation, in the FIGURE of the accompanying drawing.

FIG. 1 is a graph illustrating the weight loss results of Example 5.

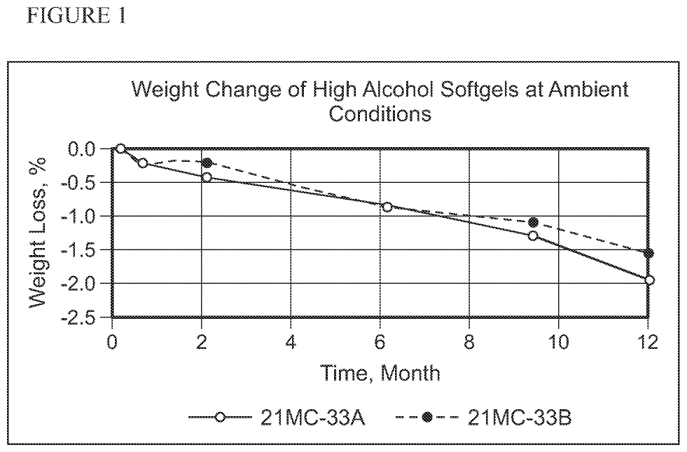

### DETAILED DESCRIPTION

The detailed description set forth below is intended to merely as a description of the presently preferred embodiments of the invention, and is not intended to represent the only form in which the present invention may constructed or utilized. The description sets forth the functions, means, and methods of implementing the invention in connection with the illustrated embodiments. It is to be understood, however, that the same or equivalent functions and features may be accomplished by different embodiments that are also intended to be encompassed within the spirit and scope of the claims.

As used herein, the singular forms “a,” “an,” and “the” include plural references unless the context clearly indicates otherwise. Thus, for example, reference to “an active pharmaceutical ingredient” includes a single active pharmaceutical ingredient as well as a mixture of two or more different active pharmaceutical ingredients, and reference to an “excipient” includes a single excipient as well as a mixture of two or more different excipients, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±5%, such that “about 10” would include from 9.5 to 10.5.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

As used herein, the terms “active agent,” “active ingredient,” “active pharmaceutical ingredient,” “API,” and “drug” refer to any material that is intended to produce a therapeutic, prophylactic, or other intended effect, whether or not approved by a government agency for that purpose. These terms with respect to specific agents include all pharmaceutically active agents, all pharmaceutically acceptable salts thereof, complexes, stereoisomers, crystalline forms, co-crystals, ether, esters, hydrates, solvates, and mixtures thereof, where the form is pharmaceutically active.

As used herein, “shell” or “shell composition” refers to the shell of a softgel capsule which encapsulates a fill material.

As used herein, “fill material” or “fill” refers to the composition that is encapsulated by the shell.

As used herein, “ambient conditions” refers to a temperature range from 15.0° C. (59° F.) to 30.0° C. (86° F.), and a relative humidity range of 20% to 50%.

In certain embodiments, the present invention is directed to a softgel capsule that could be used with a fill having a high alcohol content (e.g., greater than about 20%). In certain embodiments, the softgel capsule may be spherical, oval, oblong or twist-off. In certain embodiments, after 30 days, 90 days, 6 months, 9 months or 12 months storage at ambient conditions, the softgel capsule has a weight loss change of less than about 10%.

In some embodiments, the shell may include a film forming polymer and optionally a plasticizer. The film forming polymer may be an animal derived polymer, non-animal derived polymer, or a combination thereof. The animal derived polymer may include gelatin. The gelatin in the shell composition may include, but is not limited to, Type A gelatin, Type B gelatin, a hide gelatin, a fish gelatin, porcine gelatin and/or a bone gelatin used alone or in combination. In an embodiment, the gelatin is Type A medium to high Bloom gelatin. In an embodiment, the gelatin is Type B medium to high Bloom gelatin. Medium Bloom is when the Bloom is from about 70 grams to about 160 grams. High Bloom is when the Bloom is about 175 grams or above, or from about 175 grams to about 300 grams. In one embodiment, the gelatin is a 250 Bloom gelatin. In another embodiment, there is only one type of gelatin. In yet another embodiment, the gelatin is a combination of at least two types of gelatins. The non-animal derived polymer may include carrageenan.

The plasticizer may be glycerol, glycerin, sorbitol, a polyethylene sorbitan monooleate or a combination thereof. Other suitable plasticizers may include, but not be limited to, sugar alcohol plasticizer such as isomalt, maltitol, xylitol, erythritol, adonitol, dulcitol, pentaerythritol, or mannitol: or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly (propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

The shell may also include additional components. The additional components may include a thickening agent, buffer, water, or a combination thereof. The shell may further include pullulan.

In the shell composition, gelatin may be included in an amount from about 35 wt. % to about 75 wt. %. In some embodiments, gelatin may be in included in an amount from about 40 wt % to about 70 wt %, about 45 wt % to about 65 wt %, or about 50 wt % to about 60 wt %. In some embodiments, gelatin may be included in an amount of at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, or at least about 75 wt %.

In the shell composition, a sorbitol solution or a sorbitol sorbitan solution, POLYSORB® 85/70/00, or Sorbitol Special® may be included in an amount of from about 5 wt. % to about 35 wt. %, or about 5 wt. % to about 25 wt. %, or about 10 wt % to about 30 wt %, or about 15 wt % to about 25 wt %, or about 18 wt. % to 35 wt. %, or about 20 wt % to about 30 wt %. Glycerin (e.g., glycerin anhydrous) may also be in the shell composition in an amount from about 10 wt. % to about 40 wt. %, or about 15 wt. % to about 40 wt. %, or about 10 wt. % to about 20 wt. %, or about 10 wt % to about 30 wt %, or about 15 wt % to about 30 wt %, or about 15 wt % to about 25 wt %. Sodium alginate may also be included in the shell composition in an amount from about 1 wt. % to about 10 wt. %, or about 2 wt % to about 9 wt %, or about 3 wt % to about 8 wt %, or about 4 wt % to about 7 wt %, or about 5 wt % to about 6 wt %. The shell composition may also include purified water in an amount from about 8 wt. % to about 25 wt. %, or about 8 wt. % to about 20 wt. %, or about 10 wt % to about 20 wt %, or about 12 wt % to about 20 wt %, or about 10 wt % to about 15 wt %.

In some embodiments, the amount of sorbitol solution or sorbitol sorbitan solution in the shell composition may be at least about 5 wt %, at least about 10 wt %, at least about 15 wt %, at least about 20 wt %, at least about 25 wt %, at least about 30 wt %, or at least about 35 wt %. In some embodiments, the amount of glycerin (e.g. glycerin anhydrous) in the shell composition may be at least about 10 wt %, at least about 15 wt %, at least about 20 wt %, at least about 25 wt %, at least about 30 wt %, at least about 35 wt %, or at least about 40 wt %. In some embodiments, the amount of sodium alginate may be included in the shell composition in an amount of at least about 1 wt %, at least about 2 wt %, at least about 3 wt %, at least about 4 wt %, at least about 5 wt %, at least about 6 wt %, at least about 7 wt %, at least about 8 wt %, at least about 9 wt %, or at least about 10 wt %. In some embodiments, the amount of purified water may be at least about 8 wt %, at least about 10 wt %, at least about 13 wt %, at least about 15 wt %, at least about 18 wt %, at least about 20 wt %, at least about 22.5 wt %, or at least about 25 wt %.

The thickening/film forming agent may be a starch, a starch derivative or a modified starch. The starch or starch derivative may be hydroxypropylated tapioca starch, hydroxypropylated corn starch, potato starch, or pregelatinized modified corn starches. The modified starch includes such starches as hydroxypropylated starches, acid thinned starches and the like. In general, modified starches are products prepared by chemical treatment of starches, for example, acid treatment starches, enzyme treatment starches, oxidized starches, cross-bonding starches, and other starch derivatives

The buffer and/or an alkalizing agent may be, but is not limited to, ammonium hydroxide, sodium hydroxide, sodium carbonate, sodium citrate, trisodium phosphate and/or disodium phosphate. In one embodiment, the buffer is disodium phosphate.

In some embodiments, the fill composition of the softgel capsule includes at least about 20 wt. % alcohol, at least about 30 wt. % alcohol, at least about 40 wt. % alcohol, at least about 50 wt. % alcohol, at least about 60 wt. % alcohol, or at least about 75 wt. % alcohol. In some embodiments, the alcohol is included in an amount from about 20 wt % to about 75 wt %, about 25 wt % to about 70 wt %, about 30 wt % to about 65 wt %, about 35 wt % to about 60 wt %, about 40 wt % to about 55 wt %, or about 45 wt % to about 50 wt %, or any range or sub range therein.

The alcohol in the fill composition may be ethanol, ethyl alcohol or isopropanol. In some embodiments, the ethanol may be ethanol 40%, ethanol 50%(100 Proof), ethanol 60%, ethanol 65%, ethanol 70%, ethanol 75%, ethanol 80%, or ethanol 100%(200 Proof). In some embodiments, ethyl alcohol may be ethyl alcohol 70%, ethyl alcohol 75%, ethyl alcohol 80%, ethyl alcohol 85%, or ethyl alcohol 90%. In some embodiments, the isopropanol may be isopropanol 60%, isopropanol 65%, isopropanol 70%, isopropanol 75%, isopropanol 80%, or isopropanol 85%.

The fill composition of the softgel composition may further include additional ingredients. Those additional ingredients may be water, acrylates copolymer, xanthan gum, Carbomer, glycerin, butylene glycol, aminomethyl propanol, aloe barbaensis leaf extract, an active pharmaceutical ingredient, or a combination thereof.

In certain embodiments the invention is directed to a softgel capsule comprising a shell comprising a film forming polymer and a plasticizer, and a fill composition comprising at least about 20 wt. % alcohol, wherein after 30 days, 90 days, 6 months, 9 months, or 12 months storage at ambient conditions, the softgel capsule has a weight loss change of less than about 10%.

In certain embodiments of the invention, after 30 days storage at ambient conditions, the softgel capsule has a weight loss change of less than about 8%, less than about 5%, less than about 3%, less than about 2%, less than about 1%, less than about 0.5% or less than about 0.25%.

In certain embodiments of the invention, after 2 months storage at ambient conditions, the softgel capsule has a weight loss change of less than about 8%, less than about 5%, less than about 3%, less than about 1%, less than about 0.5%, or less than about 0.25%.

In certain embodiments of the invention, after 6 months storage at ambient conditions, the softgel capsule has a weight loss change of less than about 8%, less than about 5%, less than about 3%, less than about 1%, less than about 0.5%, or less than about 0.25%.

In certain embodiments of the invention, after 9 months storage at ambient conditions, the softgel capsule has a weight loss change of less than about 8%, less than about 5%, less than about 3%, less than about 2%, less than about 1%, less than about 0.5%, or less than about 0.25%.

In certain embodiments of the invention, after 12 months storage at ambient conditions, the softgel capsule has a weight loss change of less than about 8%, less than about 5%, less than about 3%, less than about 2%, less than about 1%, less than about 0.5%, or less than about 0.25%.

In certain embodiments of the invention, the storage at ambient conditions is in a closed container.

In other embodiments of the invention, the storage at ambient conditions is in an open container.

In certain embodiments of the invention the film forming polymer comprises an animal derived polymer or a non-animal derived polymer.

In certain embodiments of the invention, the animal derived polymer comprises gelatin.

In certain embodiments of the invention, the non-animal derived polymer comprises alginate, carrageenan, pullulan or a combination thereof.

In certain embodiments of the invention, the alcohol comprises ethanol, isopropanol or a combination thereof.

In certain embodiments of the invention, the shell further comprises a thickening agent.

In certain embodiments of the invention, the shell further comprises a buffer.

In certain embodiments of the invention, the shell further comprises a gelling salt. In certain embodiments, the gelling salt may be calcium chloride, sodium chloride, potassium chloride, sodium citrate, calcium acetate, calcium citrate, calcium gluconate, calcium lactate, sodium phosphate, or potassium phosphate.

In certain embodiments of the invention, the shell further comprises water.

In certain embodiments of the invention, the plasticizer comprises glycerin, sorbitol, a polyethylene sorbitan monooleate or a combination thereof.

In certain embodiments of the invention, the shell further comprises pullulan.

In certain embodiments of the invention, the thickening/film forming agent comprises a starch or a starch derivative.

In certain embodiments of the invention, the buffer comprises sodium phosphate.

In certain embodiments of the invention, the shell contains from about 0.1 gram of fill composition to about 2 gram of fill composition. In other embodiments, the shell may contain from about 0.1 gram to about 1.75 gram, or 0.25 gram to about 1.5 gram, or about 0.5 gram to about 1.25 gram, or about 0.75 gram to about 1 gram of fill composition. In some embodiments, the shell may contain at least about 0.1 gram of fill composition, at least about 0.25 gram of fill composition, at least about 0.5 gram of fill composition, at least about 0.75 gram of fill composition, at least about 1 gram of fill composition, at least about 1.25 gram of fill composition, at least about 1.5 gram of fill composition, at least about 1.75 gram of fill composition, or at least about 2 gram of fill composition.

In certain embodiments of the invention, the fill composition further comprises water, acrylates copolymer, xanthan gum, glycerin, butylene glycol, aminomethyl propanol, aloe barbadensis leaf extract, or a combination thereof.

In certain embodiments of the invention, after 3 months, 6 months or 12 months, the softgel capsule has a weight loss change of less than about 10%. In some embodiments of the invention, after 1 month, 2 months, 3 months, 6 months, 9 months or 12 months, the softgel capsule has a weight loss change of less than about 10%, less than about 9%, less than about 8%, less than about 7%, less than about 6%, less than about 5%, less than about 4%, less than about 3%, less than about 2%, less than about 1%, less than about 0.5%, or less than about 0.25%.

In certain embodiments of the invention, the softgel may be spherical, oval, oblong or twist-off.

In certain embodiments of the invention, the softgel further comprises an active agent.

In certain embodiments of the invention, the active agent is solubilized in the alcohol.

In certain embodiments of the invention, the capsule disintegrates in less than about 60 minutes, less than about 45 minutes, less than about 30 minutes, less than about 20 minutes, less than about 10 minutes, or less than about 5 minutes in a gastric environment based on a disintegration test performed in a USP Apparatus II with paddles at a speed of 50 rpm in pH 1.2 buffer.

In certain embodiments of the invention, the capsule disintegrates in at least about one hour, at least about two hours, at least about three hours, at least about four hours, or at least about five hours in a basic medium based on a disintegration test performed in a basket-rack assembly NT-40H model apparatus in a 1000 ml beaker at about 37° C.±2° C.

In certain embodiments of the invention, the shell comprises an enteric material.

In certain embodiments of the invention, the capsule dissolves/disintegrates in at least about 15 minutes, at least about 30 minutes, at least about one hour, at least about two hours, at least about three hours, at least about four hours, or at least about five hours in an acidic medium based on a dissolution/disintegration test performed in a USP Apparatus II with paddles at a speed of 50 rpm in 0.1N HCl, optionally with pepsin, or in simulated gastric fluid.

In certain embodiments of the invention, the capsule dissolves/disintegrates in after at least about 10 minutes, after at least about 15 minutes, after at least about 20 minutes, after at least about 25 minutes, after at least about 30 minutes, after at least about 35 minutes, after at least about 40 minutes, or from any of about 10 minutes, about 15 minutes, about 20 minutes, about 25 minutes or about 30 minutes up to any of about 35 minutes, about 40 minutes, about 45 minutes, about 50 minutes, about 55 minutes, about 60 minutes, about 75 minutes, or about 90 minutes in an intestinal environment based on a dissolution/disintegration test performed in a USP Apparatus II with paddles at a speed of 50 rpm in pH 6.8 phosphate buffer, optionally with pancreatin, or in simulated gastric fluid.

In certain embodiments, the invention is directed to a method of treating a disease or condition comprising administering a softgel as disclosed herein to a subject or patient in need thereof.

In certain embodiments, the invention is directed to a method of preparing a softgel as disclosed herein comprising encapsulating a fill composition comprising at least about 20 wt. % alcohol in a shell comprising a film forming polymer and a plasticizer.

In certain embodiments, the invention is directed to a method of sanitizing a surface comprising dispensing an alcohol composition from a softgel as disclosed herein and applying the composition to the surface.

In certain embodiments, the surface is the skin of a subject (e.g., on one or more hands).

In certain embodiments, the surface is on a non-living object (e.g., a counter surface).

### Alternate Embodiments

In certain embodiments, the shell comprises a pH dependent polymer that solubilizes at a pH of less than about 5.5, less than about 5.0, less than about 4.5 or less than about 4.0.

In certain embodiments, the pH dependent polymer is an acrylic polymer or a cellulosic polymer.

In certain embodiments, the pH dependent polymer is an acrylic polymer such as an amino methacrylate copolymer.

In certain embodiments, the shell composition comprises the pH dependent polymer in an amount (w/w) from about 1% to about 90%, about 2% to about 60%, from about 5% to about 50%, from about 10% to about 40% or about 15% to about 35%. In other embodiments, the shell composition may comprise the pH dependent polymer in an amount (w/w) of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or any sub-range or single concentration value therein, with all wt. % being based on the total weight of the shell composition

In certain embodiments, the shell composition comprises the gelatin in an amount (w/w) from about 5% to about 75%, from about 10% to about 60%, from about 15% to about 50% or about 20% to about 40%. In some embodiments, the shell composition may comprise the gelatin in an amount (w/w) of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% or any sub-range or single concentration value therein, with all wt. % being based on the total weight of the shell composition.

In certain embodiments, the shell composition further comprises a solubilizing agent such as an organic acid, e.g., oxalic acid, malonic acid, succinic acid, glutaric acid, adipic acid, fumaric acid, maleic acid, phthalic acid, isophthalic acid, terephthalic acid, formic acid, acetic acid, propionic acid, butyric acid, valeric acid, cinnamic acid, lactic acid, benzoic acid, salicylic acid, gallic acid or toluic acid. In a particular embodiment, the organic acid is lactic acid.

In certain embodiments, the shell composition comprises the solubilizing agent in an amount (w/w) from about 0.0001% to about 1%, from about 0.001% to about 0.5%, or about 0.005% or about 0.1%. In some embodiments, the shell composition may comprise the solubilizing agent in an amount (w/w) from about 0.01% to about 0.5%, from about 0.1% to about 1%, or any range or value therein. In some embodiments, the shell composition may comprise the solubilizing agent in an amount of at least about 0.0001%, at least about 0.001%, at least about 0.005%, at least about 0.01%, at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, or at least about 1% or any sub-range or single concentration value therein, with all wt. % being based on the total weight of the shell composition.

In certain embodiments, the shell composition further comprises a plasticizer, e.g., in an amount (w/w) of about 0.1 wt % to about 50 wt %, about 5% to about 45%, about 10% to about 40% or about 15% to about 35%. In some embodiments, the shell composition may include a plasticizer in an amount of at least about 0.1 wt %, at least about 1 wt %, at least about 2.5 wt %, at least about 5 wt %, at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, or any sub-range or single concentration value therein, with all wt. % being based on the total weight of the shell composition.

In certain embodiments, the plasticizer is selected from glycerol, glycerin, sorbitol solution, sorbitol sorbitan solution or combinations thereof.

In certain embodiments, the gelatin is selected from Type A gelatin, Type B gelatin or mixtures thereof.

In certain embodiments, the gelatin is selected from the group consisting of fish gelatin, hide gelatin, bone gelatin or mixtures thereof.

In certain embodiments, the liquid medium is water, a polyol, a glycol, an alcohol or a combination thereof. The polyol may be glycerol: the glycol may be polyethylene glycol and the alcohol may be ethanol.

In certain embodiments, the fill composition comprises the alcohol in an amount of at least 20%, at least 30%, at least 40%, at least 50%, at least 60%, at least 70%, at least 80%, at least 90%, at least 95% or at least 99%. In certain embodiments, the fill composition comprises the alcohol in an amount of from 20% to 50%, about 50% to about 70% or about 70% to about 95%.

In certain embodiments, the capsule disintegrates in less than about 60 minutes, less than about 45 minutes, less than about 30 minutes, less than about 20 minutes, less than about 10 minutes, or less than about 5 minutes in a gastric environment based on a disintegration test performed in a USP Apparatus II with paddles at a speed of 50 rpm in pH 1.2 buffer.

In certain embodiments, the capsule disintegrates in at least about one hour, at least about two hours, at least about three hours, at least about four hours, or at least about five hours in a basic medium based on a disintegration test performed in a basket-rack assembly NT-40H model apparatus in a 1000 ml beaker at about 37° C.±2° C.

Suitable fill materials may comprise additional fill components such as flavoring agents, sweetening agents, coloring agents and fillers or other pharmaceutically acceptable excipients or additives such as synthetic dyes and mineral oxides.

In some embodiments, the fill material (with or without the active agent) within a dosage form according to the disclosure has a pH of greater than about 3. For example, the pH of the fill material is greater than about 3, greater than about 4, greater than about 5, greater than about 6, greater than about 7, greater than about 8, greater than about 9, or about 4 to about 14, about 4 to about 6, about 5 to about 7, about 6 to about 8, about 7 to about 9, about 8 to about 10, about 9 to about 11, about 10 to about 12, about 11 to about 13, about 12 to about 14, about 10 to about 14, about 10 to about 13, about 10 to about 12, or any individual pH or sub-range within these ranges.

In an embodiment, the gelatin in the shell composition may include Type A gelatin, Type B gelatin, a hide or skin gelatin and/or a bone gelatin used alone or in combination. In one embodiment, the gelatin is a 250 Bloom gelatin. In another embodiment, there is only one type of gelatin. In yet another embodiment, the gelatin is a combination of at least two types of gelatins. In an embodiment, the amount of gelatin in the shell composition is about 5 wt. % to about wt. 90%, about 10 wt. % to about 80 wt. %, about 20 wt. % to about 80 wt. %, about 40 wt. % to about 80 wt. %, or from about 45 wt. % to about 75 wt. %, or from about 50 wt. % to about 70 wt. %. In some embodiments, the amount of gelatin may be at least about 5 wt %, at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, or at least about 90 wt % or any sub-range or single concentration value therein, with all wt. % being based on the total weight of the shell composition.

In one embodiment, the shell composition may comprise dextrose. In an embodiment, the amount of dextrose in the pH dependent capsule shell composition is about 0.005 wt. % or about 0.01 wt. % to about 4 wt. %, or from about 0.1 wt. % or about 0.15 wt. % to about 3 wt. %, or from about 0.15 wt. % or about 0.2 wt. % to about 2 wt. %, or from about 0.1 wt. % to about 0.2 wt. %. In some embodiments, the amount of dextrose in the pH dependent capsule shell composition may be at least about 0.005 wt %, at least about 0.01 wt %, at least about 0.1 wt %, at least about 0.15 wt %, at least about 0.2 wt %, at least about 0.5 wt %, at least about 1 wt %, at least about 2 wt %, at least about 3 wt % or at least about 4 wt % or any sub-range or single concentration value therein, with all wt. % being based on the total weight of the shell composition.

In an embodiment, the plasticizer in the shell composition may include glycerol, glycerin, sorbitol solution, sorbitol sorbitan solution and combinations thereof. Other suitable plasticizers may include, but not be limited to, sugar alcohol plasticizer such as isomalt, maltitol, xylitol, erythritol, adonitol, dulcitol, pentaerythritol, or mannitol: or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In an embodiment, the amount of plasticizer in the shell composition is about 0.1 wt. % to about 50 wt. %, about 15 wt. % to about 40 wt. %, or from about 20 wt. % to about 35 wt. %, or from about 25 wt. % to about 30 wt. %. In some embodiments, the amount of plasticizer may be at least about 0.1 wt %, at least about 1 wt %, at least about 5 wt %, at least about 10 wt %, at least about 15 wt %, at least about 20 wt %, at least about 25 wt %, at least about 30 wt %, at least about 35 wt %, at least about 40 wt %, at least about 45 wt %, or at least about 50 wt % or any sub-range or single concentration value therein, with all wt. % being based on the total weight of the shell composition.

In an embodiment, the shell composition may optionally comprise additional agents such as coloring agents, flavorings agents, sweetening agents, fillers, antioxidants, diluents, pH modifiers or other pharmaceutically acceptable excipients or additives such as synthetic dyes and mineral oxides.

Exemplary suitable coloring agents may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

Exemplary suitable flavoring agents may include, but not be limited to, “flavor extract” obtained by extracting a part of a raw material, e.g., animal or plant material, often by using a solvent such as ethanol or water: natural essences obtained by extracting essential oils from the blossoms, fruit, roots, etc., or from the whole plants.

Additional exemplary flavoring agents that may be in the dosage form may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

Exemplary sweetening agents may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet R and Equal R), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin: monoammonium glycyrrhizinate (sold under the trade name MagnaSweet®): Stevia rebaudiana (Stevioside), natural intensive sweeteners, such as Lo Han Kuo, polyols such as sorbitol, mannitol, xylitol, erythritol, and the like.

Encapsulation of the fill material can be accomplished in any conventional manner. As an example, a rotary die encapsulation machine may be used.

According to an embodiment, a pH dependent softgel capsule is prepared by the process comprising the steps of: (a) preparing the fill material as disclosed herein, said fill material optionally comprising at least one pharmaceutically active ingredient; and (b) encapsulating the fill material of step (a) in a shell composition disclosed herein. The encapsulation process according to step (b) may further comprise a sub-step of preparing the shell composition by, for example, admixing a film forming polymer and plasticizer.

### Non-Animal Film Forming Agents

In certain embodiments, the shell composition comprises a non-animal film forming agents, e.g., carrageenan, pullulan, starch, pregelatinized starch, xanthan gum, agar, pectin, alginate, sugar, sugar derived alcohol, monosaccharides, disaccharides, oligosaccharides, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, arbopol, silicon dioxide, curdlan, furcelleran, albumin (e.g., egg or lacto derived), soy protein, chitosan, guaic gum, tamarind seed polysaccharide, glucomannan, chitin, pluran, inulin, cyclodextrin, or a combination thereof.

The carrageenan can be at least one of iota carrageenan, kappa carrageenan and lambda carrageenan.

The starch can be modified starch or native starch, sweet potato starch, potato starch, corn starch, tapioca starch, pea starch, hydroxy propylated starch, hydroxyalkylated starch, acid-treated starch, dextrin, high amylose non-modified corn starch, modified waxy maize starch, non-granular starch, modified high amylose corn starch, pregelatinized rice flour and a combination thereof. As used herein and in the claims, the term “modified starch” includes such starches as hydroxypropylated starches, acid thinned starches and the like. In general, modified starches are products prepared by chemical treatment of starches, for example, acid treatment starches, enzyme treatment starches, oxidized starches, cross-bonding starches, and other starch derivatives. It is preferred that the modified starches be derivatized wherein side chains are modified with hydrophilic or hydrophobic groups to thereby form a more complicated structure with a strong interaction between side chains.

In certain embodiments, the non-animal film forming agent is in the shell composition in an amount, e.g., of about 2 wt. % to about 20 wt. %, about 2 wt. % to about 15 wt. %, about 2 wt. % to about 40 wt. %, about 10 wt. % to about 80 wt. %, or about 15 wt. % to about 75 wt. %, or about 20 wt. % to about 70 wt. %, or about 25 wt. % to about 60 wt. %, or about 25 wt. % to about 45 wt. %, or about 20 wt. % to about 35 wt. %, or about 30 wt. % to about 40 wt. %, or about 32 wt. %, or about 35 wt. %, or about 38 wt. %, or any sub-range or single concentration value therein, with all wt. % being based on the total weight of the shell composition. In some embodiments, the non-animal film forming agent is in the shell composition in an amount of at least about 2 wt %, at least about 5 wt %, at least about 10 wt %, at least about 15 wt %, at least about 20 wt %, at least about 25 wt %, at least about 30 wt %, at least about 32 wt %, at least about 35 wt %, at least about 38 wt %, at least about 40) wt %, at least about 45 wt %, at least about 50 wt %, at least about 55 wt %, at least about 60 wt %, at least about 65 wt %, at least about 70 wt %, at least about 75 wt %, at least about 80 wt %, or any sub-range or single concentration value therein, with all wt. % being based on the total weight of the shell composition.

In one embodiment, the non-animal gelling agent includes carrageenan and does not include starch (or modified starch). In one embodiment, the softgel shell composition is substantially free or free of starch (or modified starch).

### Enteric Materials

In certain embodiments, the shell composition comprises an enteric polymeric material, e.g., acrylic and methacrylic acid polymers, which may be available under the tradename EUDRAGIT®, Kollicoat® MAE 100 P, and other acid insoluble polymers, e.g., methyl acrylate-methacrylic acid copolymers. Other acid insoluble polymers include, without limitation, cellulose acetate succinate, cellulose acetate phthalate, cellulose acetate butyrate, hydroxypropyl methyl cellulose phthalate, hydroxy propyl methyl cellulose acetate succinate (hypermellose acetate succinate), polyvinyl acetate phthalate (PVAP), alginic acid salts such as sodium alginate and potassium alginate, stearic acid, and shellac. Other enteric materials include pectin, pectin derivatives, gellan gum and gellan gum derivatives.

### Active Agents

Any pharmaceutically active ingredient may be used for purposes of the present invention, alcohol soluble. Suitable pharmaceutically active ingredients include, without limitation, analgesics and anti-inflammatory agents, antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, oral vaccines, proteins, peptides and recombinant drugs, sex hormones and contraceptives, spermicides, stimulants, and combinations thereof.

In some embodiments, the active pharmaceutical ingredient may be selected, without limitations, from the group consisting of dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

According to certain embodiments, active agents may include lipid-lowering agents including, but not limited to, statins (e.g., lovastatin, simvastatin, pravastatin, fluvastatin, atorvastatin, rosuvastatin, and pitavastatin), fibrates (e.g, clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical active agents may include, but are not limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sequioxide, glandular products, glucosamine HCl, glucosamine sulfate, hydroxyl methyl butyrate, immunoglobulin, lactic acid, L-Carnitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplement active agents may include vitamins, minerals, fiber, fatty acids, amino acids, herbal supplements or a combination thereof.

Suitable vitamin active agents may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Suitable herbal supplement active agents may include, but are not limited to, the following: arnica, bilberry, black cohosh, cat's claw, chamomile, echinacea, evening primrose oil, fenugreek, flaxseed, feverfew, garlic, ginger root, ginko biloba, ginseng, goldenrod, hawthorn, kava-kava, licorice, milk thistle, psyllium, rauowolfia, senna, soy bean, St. John's wort, saw palmetto, turmeric, valerian.

Minerals active agents may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Examples of other possible active agents include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib. Cox-2 inhibitors, diclofenac, benoxaprofen, flurbiprofen, fenoprofen, flubufen, indoprofen, piroprofen, carprofen, oxaprozin, pramoprofen, muroprofen, trioxaprofen, suprofen, aminoprofen, fluprofen, bucloxic acid, indomethacin, sulindac, zomepirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, faislamine, fenbufen, fenoprofen, flurbiprofen, ibuprofen, indometacin, ketoprofen, ketorolac, lornoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin, pharmaceutically acceptable salts thereof and mixtures thereof) and acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent that may also be a benzodiazepine, barbiturate, stimulants, or mixtures thereof. The term “benzodiazepines” refers to a benzodiazepine and drugs that are derivatives of a benzodiazepine that are able to depress the central nervous system. Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2, 4, 6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

The dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like: organic acid salts such as formate, acetate, trifluoroacetate, maleate, tartrate and the like: sulfonates such as methanesulfonate, benzenesulfonate, p-toluenesulfonate, and the like: amino acid salts such as arginate, asparginate, glutamate and the like, and metal salts such as sodium salt, potassium salt, cesium salt and the like: alkaline earth metals such as calcium salt, magnesium salt and the like: organic amine salts such as triethylamine salt, pyridine salt, picoline salt, ethanolamine salt, triethanolamine salt, dicyclohexylamine salt, N,N′-dibenzylethylenediamine salt and the like.

### EXAMPLES

The present invention will now be more fully described with reference to the accompanying examples. It should be understood, however, that the following description is illustrative only and should not be taken in any way as a restriction of the invention.

### Example 1

Alginate/gelatin shell hand sanitizer softgels were prepared. The shell composition included glycerin anhydrous, sodium alginate, dextrose anhydrous, gelatin and purified water. The shell composition is presented in Table 1 below. The air filled softgels were dried and one gram of hand sanitizer was injected into the air filled softgels, and the injection hole was sealed using the same gel mass.

TABLE 1
Shell Composition of Example 1

| Ingredients | Amount in wt. % |
|---|---|
| Gelatin | 35-70 |
| Sorbitol sorbitan solution (15-25% water | 5-25 |
| content) POLYSORB ® | |
| Glycerin anhydrous | 15-40 |
| Sodium Alginate | 1-10 |
| Purified water | 8-20 |

The ingredients and formulation of the hand sanitizer is presented in Table 2 below.

TABLE 2
List of Ingredients and Formulation in Hand Sanitizer

| Ingredients | Amount in wt. % |
|---|---|
| Alcohol | 75.00 |
| Water (Aqua) | 17.35 |
| Acrylates Copolymer | 5.00 |
| Xanthan gum | 1.00 |
| Glycerin | 0.80 |
| Butylene Glycol | 0.50 |
| Aminomethyl Propanol | 0.30 |
| Aloe Barbadensis Leaf Extract | 0.05 |

The filled softgels were then stored into glass vials, half of them with lids, i.e. closed containers, and the other half without lids, i.e. open containers. The weight and physical changes of the softgels were monitored for a period of 15 days. It was found that the softgels stored in both open containers and closed containers experienced weight loss. The results of the weight loss are presented in Table 3.

TABLE 3
Weight Loss of Alginate/Gelatin Shell Hand Sanitizer Softgels

| | Weight Loss (%) | |
|---|---|---|
| | Hold Time (Days) | |
| Sample | 7 | 15 |
| 5% Alginate Gelatin Capsule (Open Vial) | −46.76% | −48.09% |
| 5% Alginate Gelatin Capsule (Closed) | −9.44% | −13.77% |

The weight loss of alginate/gelatin shell hand sanitizer softgels in open vials was significantly higher than that of alginate/gelatin shell hand sanitizer softgels in closed vials. This indicated that the alginate/gelatin shell was quite permeable to alcohol and water vapor. The inventors suspect, without being limited to a theory, this could be due to the use of glycerin in the gel mass.

### Example 2

Non-animal polymer shell hand sanitizer softgels were prepared. The shell composition included modified starch, carrageenan, sodium phosphate, POLYSORB® 85/70/00, pullulan and purified water. The shell composition is presented in Table 4 below. The air filled softgels were dried and one gram of hand sanitizer was injected into the air filled softgels, and the injection hole was sealed using the same gel mass.

TABLE 4
Shell Composition of Example 2

| Ingredients | Amount in wt. % |
|---|---|
| Carrageenan | 5-20 |
| Pullulan | 2-15 |
| Starch | 20-45 |
| Sorbitol sorbitan solution (15-25% water | 18-35 |
| content) POLYSORB ® | |
| Glycerin anhydrous | 10-20 |
| Sodium phosphate | 0.5-2.0 |
| Purified Water | 8-25 |

The filled softgels were then stored into glass vials, half of them with lids, i.e. closed containers, and the other half without lids, i.e. open containers. The weight and physical changes of the softgels were monitored for a period of 30 days. It was found that the softgels stored in both open containers and closed containers experienced weight loss. The results of the weight loss are presented in Table 5.

TABLE 5
Capsule Weight Loss and Changes in Ethanol and Water Contents
of Non-animal Hand Sanitizer Softgel (20MC-103, Uncoated)

| | Weight Loss (Hold Time (Days) and Container Closure) | | | | |
|---|---|---|---|---|---|
| | | % (T = | % (T = | % (T = | % (T = |
| | | 14) | 30) | 14) | 30) |
| | % (T = | (Close | (Close | (Open | (Open |
| Test | 0) | Vial) | Vial) | Vial) | Vial) |
| Capsule | 0 | −4.85 | −6.04 | −11.97 | −13.59 |
| Weight Loss | | | | | |
| (%) | | | | | |
| Fill Weight | 0 | −8.69 | −10.82 | −18.84 | −21.41 |
| Loss (%) | | | | | |
| Ethanol | 75.0 | 74.57 | 74.02 | 83.40 | 84.12 |
| Content (%) | (Label) | | | | |
| Water Content | 27.07 | 22.22 | 22.69 | 12.99 | 11.93 |
| (%) | | | | | |

### Example 3

Another batch of non-animal hand sanitizer softgels was prepared. The batched was prepared with the same composition as described in Example 2. Half of this batch was filled with hand sanitizer from a commercial source without any addition of glycerin or other excipients, while the other half was filled with hand sanitizer from a commercial source with 5% glycerin anhydrous added. The theoretical fill weight of the softgels was 1000 mg per capsule. After encapsulation, the softgels were spread onto shallow trays and dried in a drying cabinet. It was observed that a portion of the hand sanitizer fill was lost during the drying process creating air bubbles in the capsules. After drying, the hand sanitizer filled capsules were inspected, washed and packaged into sealed plastic bags.

Stability studies were carried out to monitor the weight loss of the hand sanitizer filled softgels during storage under ambient conditions. Thus, the hand sanitizer filled softgels were placed into open and closed glass vials at ambient conditions. The loss in weight was checked regularly by weighing the softgels and is presented in Tables 6 and 7.

TABLE 6
Cumulative Weight Loss for Non-animal Hand Sanitizer
Capsules Filled with Pure Hand Sanitizer

| Storage | Weight Loss (Day) | | | | | | | | | |
|---|---|---|---|---|---|---|---|---|---|---|
| Condition | 0 | 1 | 2 | 3 | 6 | 7 | 9 | 14 | 21 | 30 |
| Closed | 0 | −0.15% | −0.18% | −0.18% | −0.31% | −0.35% | −0.41% | −0.56% | −0.76% | −1.01% |
| container | | | | | | | | | | |
| Open | 0 | −0.94% | −1.36% | −1.74% | −2.65% | −2.88% | −3.29% | −4.04% | −4.76% | −5.56% |
| container | | | | | | | | | | |

TABLE 7
Cumulative Weight Loss for Non-animal Hand Sanitizer Capsules
Filled with Hand Sanitizer with 5% Glycerin Added

| Storage | Weight Loss (Day) | | | | | | | | | |
|---|---|---|---|---|---|---|---|---|---|---|
| Condition | 0 | 1 | 2 | 3 | 6 | 7 | 9 | 14 | 21 | 30 |
| Closed | 0 | −0.28% | −0.42% | −0.52% | −0.97% | −1.09% | −1.31% | −1.86% | −2.62% | −3.55% |
| container | | | | | | | | | | |
| Open | 0 | −0.43% | −0.67% | −0.89% | −1.53% | −1.74% | −2.07% | −2.86% | −3.76% | −5.04% |
| container | | | | | | | | | | |

The hand sanitizer filled softgel capsules were continuously losing weight during storage at ambient conditions. However, the weight loss of those stored in a closed vial was significantly less when compared to that of those stored in an open container. Also, the rate of weight loss was gradually reduced over time.

### Example 4

A regression analysis was also performed to predict the percentage of hand sanitizer remaining after various time periods stored in both closed and open containers as shown in Tables 8 and 9.

TABLE 8
Predicted Percentage of Hand Sanitizer Remaining After Storage

| | Predicted Weight Remaining (%, Days) | | | | | |
|---|---|---|---|---|---|---|
| Prediction | 0 | 180 | 240 | 300 | 320 | 365 |
| Closed container | 100 | 94.6 | 92.8 | 91.0 | 90.4 | 89.1 |
| Open container | 100 | 62.8 | 40.7 | 16.4 | 8.0 | 0.0 |

TABLE 9
Predicted Percentage of Hand Sanitizer Remaining After Storage

| | Predicted Weight Remaining (%, Days) | | | | | |
|---|---|---|---|---|---|---|
| Prediction | 0 | 180 | 240 | 300 | 320 | 365 |
| Closed container | 100 | 80.2 | 73.6 | 67.0 | 64.8 | 59.8 |
| Open container | 100 | 40.1 | 20.2 | 0.4 | 0 | 0 |

Based on the extrapolated data it can be concluded that the hand sanitizer without glycerin softgels may retain about 90%(89.1%) of the hand sanitizer fill material for 1 year at ambient conditions. In contrast, the hand sanitizer containing glycerin may not be suitable for long term retention of the ethanol in the capsules potentially due to migration of glycerin into the shell increasing the shell permeability to loss of volatile fill contents such as ethanol. Further optimization of the fill formulation, manufacturing process parameters including rate and extent of drying and type of final bulk pack size and the head space etc., may help retain even higher than 90% fill at the end of the 1 year shelf life in a closed container.

### Example 5

A stability study was also performed. Thirty (30) high ethanol (hand sanitizer containing 70% ethanol by volume) capsules from two different sublots (21MC-33A and 21MC-33B) were packaged into closed 100 cc glass bottles to mimic the real world usage. 21MC-33A was manufactured using a commercially available hand sanitizer. 21MC-33B was manufactured using a commercially available hand sanitizer with 5% glycerin added. The bottles were stored at ambient conditions. The capsules were measured at various time intervals, up to 12 months. The results of the measurements are presented in Table 10 and the FIGURE.

TABLE 10
Weight Changes of High Ethanol Capsules at Ambient Conditions

| | Weight Loss (%) | | | | | |
|---|---|---|---|---|---|---|
| Month | 0 | 1 | 2 | 6 | 9 | 12 |
| 21MC-33A | 0.00 | −0.22 | −0.43 | −0.86 | −1.29 | −1.94 |
| 21MC-33B | 0.00 | −0.22 | −0.22 | −0.88 | −1.10 | −1.55 |

Based on the weight change data, the high ethanol capsules are stable while stored in closed containers with insignificant weight losses. Also, both sublots showed similar trends in weight losses. Thus, the effect of glycerin addition is not significant. Additionally, all the capsules had acceptable appearance without any leaking capsules or other physical defects.

Thus, the high ethanol capsules are stable while stored in closed containers with insignificant weight losses. Also, both sublots showed similar trends in weight losses. Thus, the capsules have good barrier property to ethanol and provide a viable option for encapsulation of fill materials that contain high percentage of alcohol.

1. A softgel capsule comprising:
a shell comprising a film forming polymer and a plasticizer, and
a fill composition comprising at least about 20 wt. % alcohol,
wherein after 30 days storage at ambient conditions, the softgel capsule has a weight loss change of less than about 10%.

2. The softgel capsule of claim 1, wherein the fill composition comprises at least about 30 wt. % alcohol, at least about 40 wt. % alcohol, at least about 50 wt. % alcohol, at least about 60 wt. % alcohol or at least about 75 wt. % alcohol.

3. The softgel capsule of claim 1, wherein after 30 days storage at ambient conditions, the softgel capsule has a weight loss change of less than about 8%, less than about 5%, less than about 3%, less than about 1%, less than about 0.5%, or less than about 0.25%.

4. (canceled)

5. (canceled)

6. The softgel capsule of claim 1, wherein the film forming polymer comprises an animal derived polymer or a non-animal derived polymer.

7. The softgel capsule of claim 6, wherein the animal derived polymer comprises gelatin.

8. The softgel capsule of claim 7, wherein the gelatin is included from about 35 wt. % to about 70 wt. %.

9. The softgel capsule of claim 6, wherein the non-animal derived polymer comprises alginate, carrageenan, pullulan, or a combination thereof.

10. (canceled)

11. (canceled)

12. The softgel capsule of claim 9, wherein the carrageenan is included in an amount from about 5 wt. % to about 20 wt. %.

13. The softgel capsule of claim 1, wherein the alcohol comprises ethanol, isopropanol, or a combination thereof.

14. (canceled)

15. The softgel capsule of claim 1, wherein the shell further comprises a thickening agent, a buffer, water, or a combination thereof.

16. (canceled)

17. (canceled)

18. The softgel capsule of claim 15, wherein water is included in an amount from about 8 wt. % to about 25 wt. %.

19. The softgel capsule of claim 1, wherein the plasticizer comprises glycerin, sorbitol sorbitan solution, a polyethylene sorbitan monooleate or a combination thereof.

20.-23. (canceled)

24. The softgel capsule of claim 15, wherein the thickening agent comprises a starch or a starch derivative.

25.-29. (canceled)

30. The softgel capsule of claim 1, wherein after 3 months, 6 months or 12 months, the softgel capsule has a weight loss change of less than about 10%.

31.-38. (canceled)

39. The softgel capsule of claim 1, wherein after 2 months storage at ambient conditions, the softgel capsule has a weight loss change of less than about 8%, less than about 5%, less than about 3%, less than about 1%, less than about 0.5%, or less than about 0.25%.

40. The softgel capsule of claim 1, wherein after 6 months storage at ambient conditions, the softgel capsule has a weight loss change of less than about 8%, less than about 5%, less than about 3%, less than about 1%, less than about 0.5%, or less than about 0.25%.

41. The softgel capsule of claim 1, wherein after 9 months storage at ambient conditions, the softgel capsule has a weight loss change of less than about 8%, less than about 5%, less than about 3%, less than about 2%, less than about 1%, less than about 0.5%, or less than about 0.25%.

42. The softgel capsule of claim 1, wherein after 12 months storage at ambient conditions, the softgel capsule has a weight loss change of less than about 8%, less than about 5%, less than about 3%, less than about 2%, less than about 1%, less than about 0.5%, or less than about 0.25%.

43. (canceled)

44. A method of preparing a softgel of any preceding claims comprising preparing encapsulating a fill composition comprising at least about 20 wt. % alcohol in a shell comprising a film forming polymer and a plasticizer.

45. A method of sanitizing a surface comprising dispensing an alcohol composition from a softgel of claim 1 and applying the composition to the surface.

46.-48. (canceled)
